(19) United States
(12) Patent Application Publication
(10) Pub. No.: US 2007/0083236 A1
(43) Pub. Date: Apr. 12, 2007

(54) METHODS AND DEVICES FOR TISSUE REPAIR

(75) Inventors: George J. Sikora, Bridgewater, MA (US); Raymond A. Bojarski, Attleboro, MA (US); Aaron T. Hecker, West Roxbury, MA (US)

Correspondence Address: FISH & RICHARDSON P.C.;SMITH & NEPHEW, INC., 1450 BROOKS ROAD, MEMPHIS, TN, 38116, US

(73) Assignee: SMITH & NEPHEW, INC., Memphis, TN (US)

(21) Appl. No.: 11/535,868

(22) Filed: Sep. 27, 2006

Related U.S. Application Data
(63) Continuation-in-part of application No. 11/165,551, filed on Jun. 24, 2005.

(51) Int. Cl. A61B 17/04

(52) U.S. Cl. 606/232

(57) ABSTRACT

An apparatus for tissue repair includes first and second fixation members configured to secure tissue together, a flexible coupling member coupling the first and second fixation members, a first flexible pull member attached to the first fixation member but not the second fixation member, and a second flexible pull member attached to the second fixation member but not the first fixation member. A surgical method for repairing a wound in a rotator cuff includes forming a first and second channel through tissue, advancing a first flexible member and a first fixation member coupled thereto through the first channel, advancing a second flexible member and a second fixation member coupled thereto through the second channel, and pulling a third flexible member coupling the first and second fixation members to shorten a length of the third flexible member between the first and second fixation members. A surgical method includes pulling a first implant through soft tissue of a rotator cuff cross a tear in the tissue to position the first implant on a first side of the tear and a second implant, coupled to the first implant by a flexible coupling member, on a second side of the tear with the flexible coupling member traversing the tear, and pulling the flexible coupling member to shorten a length of the flexible coupling member between the first and second implants to move the implants against the soft tissue to close the tear.

### CROSS REFERENCE TO RELATED APPLICATIONS

This application is a continuation-in-part of co-pending U.S. application Ser. No. 11/165,551, filed Jun. 24, 2005, which is incorporated herein by reference. This application relates to U.S. application Ser. No. 09/704,926, which is incorporated herein by reference.

### TECHNICAL FIELD

This invention relates to devices and methods for repairing tissue, and more particularly to devices and methods for repairing tears in rotator cuff tissue.

### BACKGROUND

On area in the body where soft tissue is surgically reattached to bone is the attachment of a rotator cuff tendon to the humerus. The rotator cuff tendons have areas of low blood supply. With an increased blood supply, a tissue, such as tendon, can repair and maintain itself better and faster. Thus, areas of poor blood supply in the rotator cuff make these tendons difficult and slow to heal following an injury, such as a tear to the supraspinatus muscle or the subscapularis muscle. In such a tear, part of the tendon is pulled away from the bone. Because of the poor blood supply, rather than attempting to allow an injured rotator cuff to heal on its own, a physician often recommends that the tendon be surgically repaired to better fix the position of the cuff to the bone to prevent further damage and improve the environment for healing. For example, the physician may attempt to fix the tendon to the bone using a fixation member such as a retainer or an anchor. One example of a fixation member is disclosed in U.S. Pat. No. 4,741,330 (the Hayhurst patent), which is incorporated herein by reference.

Other areas in the body also have tissue that can be surgically reattached to bone when torn from the bone or can be surgically repaired when a tear forms in the tissue. These areas include for example, the biceps tendon, the lateral collateral ligament in the knee, the medial collateral ligament in the knee, the meniscus in the knee, the popliteal ligament in the leg, and the labrum tendon in the knee.

Fibrous tissue wounds, such as muscle, ligament, and cartilage tears, can be repaired arthroscopically using flexible members such as sutures. Traditionally, to close a fibrous tissue wound, a surgeon would insert two suture needles into the tissue with sutures attached, thread the sutures across the wound, and then tie knots to fix the free ends of the sutures within the tissue.

To simplify the wound closure procedure and to improve fixation, various types of fixation members have been developed. One example of a fixation member in the form of a retainer is disclosed in the Hayhurst patent. In the Hayhurst patent, one end of a flexible member is fixed to a resiliently-deformable, bar-shaped retainer. The retainer is loaded into the bore of a hollow needle and deployed into or against the fibrous tissue. The surgeon then threads the flexible member across the wound and tensions a free end of the suture to pull the wound closed. When the surgeon tensions the suture, the bar in the retainer becomes oriented transversely to the suture hole, holding the suture in place.

### SUMMARY

In one general aspect, a tissue repair device includes a closed loop of multifilament flexible material. The loop is knotless and includes a contact portion in which ends of the multifilament flexible material are interwoven and melted-formed.

Implementations can include one or more of the following features. For example, the tissue repair device can include a fixation member having a structure that defines a cavity that receives at least a part of the closed loop.

The tissue repair device can include a flexible member traversing the loop. The flexible member can traverse the loop by being passed through an interior defined by the loop. The flexible member can traverse the loop by being passed through the multifilament flexible material.

The ends of the multifilament flexible material can be thermally fused together within the contact portion. The flexible member can traverse the loop by being passed through the thermally fused portion of the multifilament flexible material.

The multifilament flexible material can be made of polymer-based compound.

The flexible member can traverse the loop by being passed through the interwoven portion of the multifilament flexible material. The multifilament flexible material can be braided or twisted.

In another general aspect, a tissue repair device is made by forming a closed loop from the multifilament flexible material. The forming includes interweaving ends of the multifilament flexible material together to form a contact portion without tying the ends together in a knot, and causing the ends of the multifilament flexible material to melt in the contact portion.

Implementations can include one or more of the following features. For example, the method can also include passing at least a part of the multifilament flexible material through a cavity defined by a fixation member.

The method can include traversing a flexible member through the loop. The traversing can include passing the flexible member through an interior defined by the loop. The traversing can include passing the flexible member through the multifilament flexible material. The traversing can include passing the flexible member through the contact portion of the multifilament flexible material.

Forming the closed loop can include thermally fusing the ends of the multifilament flexible material in the contact portion. Forming the closed loop from the multifilament flexible material can include forming without applying a filler material to the ends of the flexible element.

In another general aspect, a tissue repair device includes a closed loop of multifilament flexible material, and a fixation member. The loop is knotless and includes a contact portion in which ends of the multifilament flexible material are interwoven. The fixation member has a structure that defines a cavity that receives at least a part of the closed loop.

In another general aspect, a tissue repair device includes a fixation member having a structure that defines a cavity, a multifilament flexible element, and a flexible member. The multifilament flexible element includes a part that is within the cavity, and a thermally fused end. The flexible member passes at least partially through the thermally fused end of the multifilament flexible element.

Implementations can include one or more of the following features. In particular, the multifilament flexible element includes another thermally fused end and the flexible member passes through the other thermally fused end of the multifilament flexible element.

Aspects of the device and method may include one or more of the following advantages. The ends of the multifilament flexible material are thermally fused together without the use of a filler material. The loop acts as a pulley that reduces pinching of the flexible member between the tissue and the fixation member during deployment. Additionally, the pulley design enables the flexible member to slide relative to the fixation member without being impeded by the edges of the fixation member or by the tissue when the fixation member is deployed in tissue.

In another general aspect, an apparatus for tissue repair includes first and second fixation members configured to secure tissue together, and a flexible coupling member coupling the first and second fixation members. The apparatus also includes a first flexible pull member attached to the first fixation member but not the second fixation member, and a second flexible pull member attached to the second fixation member but not the first fixation member.

Implementations can include one or more of the following features. For example, the flexible coupling member includes a slip knot and a first closed loop. Additionally, the first and second fixation members each define at least one opening, and the first closed loop of the flexible coupling member traverses the opening in one of the fixation members, and a second closed loop of flexible material traverses the opening in the other fixation member with the flexible coupling member being slidably received through the second closed loop.

The first and second pull members include closed loops, and along with the flexible coupling member, can be sutures.

In another general aspect, a surgical method for repairing a wound in a rotator cuff includes forming a first channel through tissue, advancing a first flexible member and a first fixation member coupled thereto through the first channel, forming a second channel through tissue, and advancing a second flexible member and a second fixation member coupled thereto through the second channel. The method further includes pulling a third flexible member coupling the first and second fixation members to shorten a length of the third flexible member between the first and second fixation members.

Implementations can include, for example, the first and second channels being formed through rotator cuff tissue and bone tissue.

In another general aspect, a surgical method includes pulling a first implant through soft tissue of a rotator cuff across a tear in the tissue to position the first implant on a first side of the tear and a second implant, coupled to the first implant by a flexible coupling member, on a second side of the tear with the flexible coupling member traversing the tear. The method further includes pulling the flexible coupling member to shorten a length of the flexible coupling member between the first and second implants to move the implants against the soft tissue to close the tear.

Implementations can include one or more of the following features. For example, the method includes forming a channel through the soft tissue and across the tear. In addition, pulling the first implant includes pulling a first closed loop of flexible material attached to the first implant through the channel.

In another general aspect, a method of making a tissue repair device includes passing a flexible material through an opening defined by a first fixation member, forming the flexible material into a first closed loop, traversing a flexible member through the first closed loop, traversing the flexible member through a second fixation member, and forming a second closed loop in the flexible member such that the flexible member is coupled to the second fixation member. The method further includes forming a slip knot in the flexible member.

Implementations can include, for example, coupling a first flexible pull member to the first fixation member but not the second fixation member, and coupling a second flexible pull member to the second fixation member but not the first fixation member.

In another general aspect, a tissue repair device includes a closed loop of multifilament flexible material and a fixation member defining a cavity that receives at least a part of the closed loop. The loop is knotless and includes a contact portion in which the ends of the multifilament flexible material are formed together.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A is a perspective view of a tissue repair device.

FIG. 1B is an illustration of the tissue repair device of FIG. 1A, shown mending a tear in soft tissue.

FIG. 1C is a perspective view of the tissue repair device of FIG. 1A, shown mending a tear in soft tissue.

FIG. 2 is a side cross-sectional view of a fixation member and a loop of the tissue repair device of FIG. 1A.

FIGS. 3A-3C are side perspective views showing formation of a retaining element that can be formed in the tissue repair device of FIG. 1A.

FIG. 4 is a flow chart of a procedure for forming the loop in the tissue repair device of FIG. 1A.

FIGS. 5A-5E show perspective views of the multifilament flexible material that is formed into the loop in the procedure of FIG. 4.

FIG. 6 is a perspective view of another implementation of a tissue repair device.

FIG. 7 is a perspective view of another implementation of a tissue repair device.

FIG. 8 illustrates another implementation of a tissue repair apparatus.

FIGS. 9A-9E illustrate the placement of the tissue repair apparatus of FIG. 8 in the shoulder joint to repair a torn rotator cuff.

FIG. 10 illustrates another implementation of a tissue repair apparatus.

FIG. 11 illustrates the placement of the tissue repair apparatus of FIG. 10 in the rotator cuff to repair a tear in the rotator cuff soft tissue.

### DETAILED DESCRIPTION

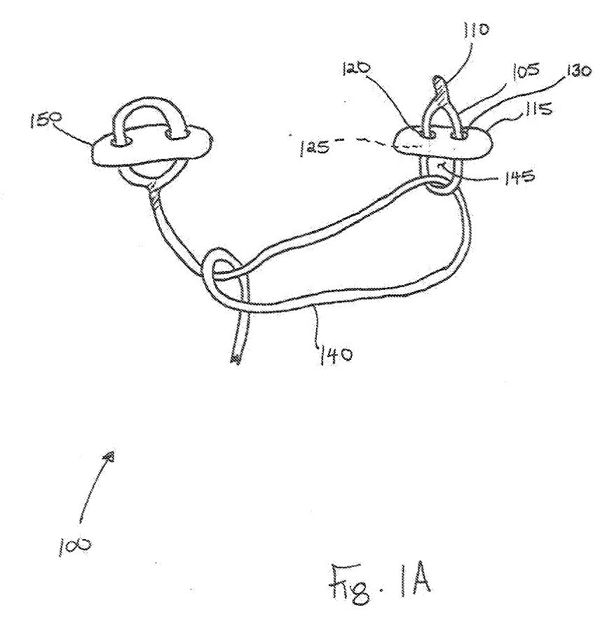

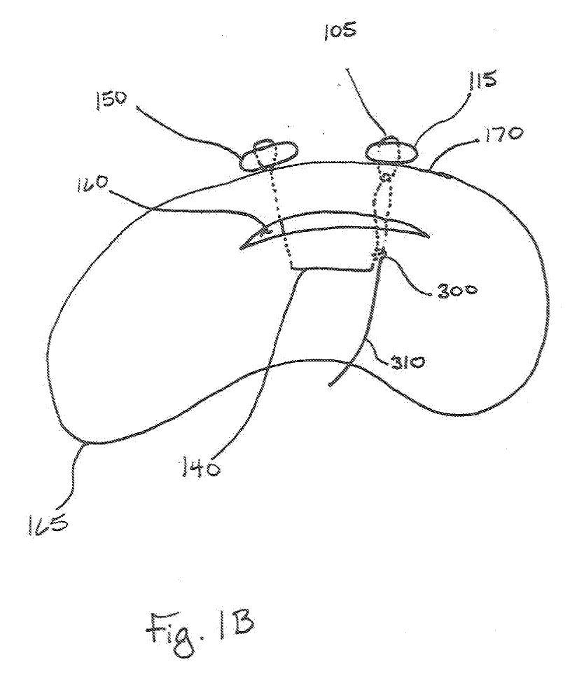

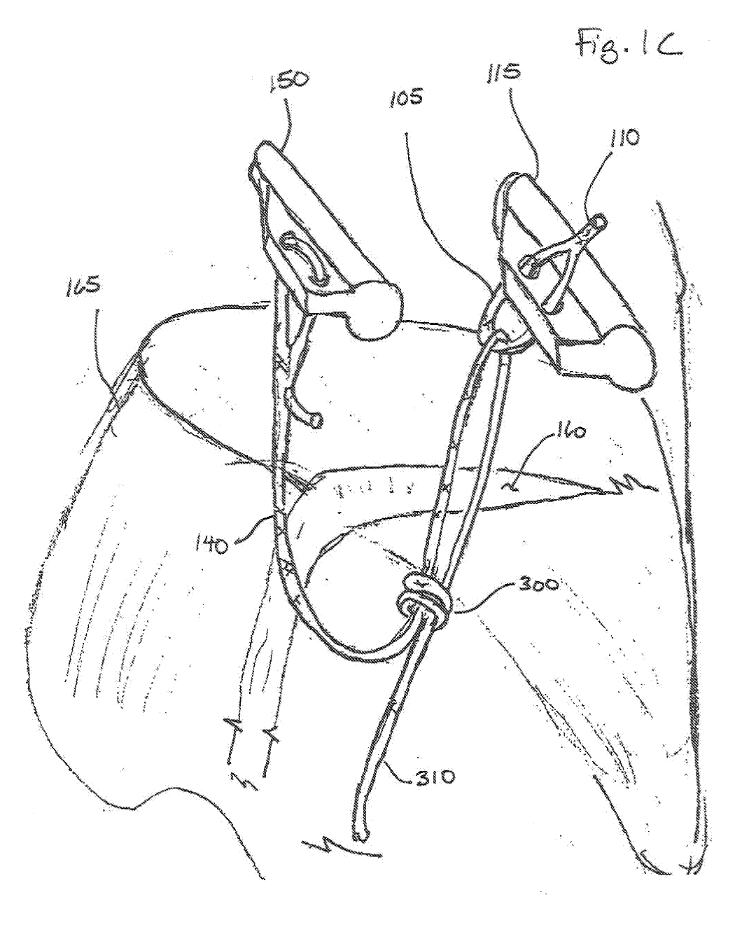

Referring to FIGS. 1A-1C and 2, a tissue repair device 100 includes a closed loop 105 of multifilament flexible material. The loop 105 is knotless, that is, the loop 105 is formed without tying ends of the multifilament flexible material together in a knot. The multifilament flexible material is a material suitable for implantation into hard or soft human tissue and it may be absorbable or nonabsorbable. The multifilament flexible material has two or more fibers or strands that are twisted, braided, or otherwise interlinked about each other. The multifilament flexible material is capable of being flexed or bent. The loop 105 is closed, with a first end of the multifilament flexible material contacts a second end of the multifilament flexible material to form a contact portion 110.

The tissue repair device 100 also includes a fixation member 115 defining a cavity 120 that receives a part 125 of the loop 105. As shown, the fixation member 115 can also include a second cavity 130 that receives another part 135 of the loop 105. The fixation member 115 can be made of any rigid material suitable for implantation into hard or soft human tissue. For example, the fixation member 115 can be made of a biocompatible plastic, a biocompatible metal, or a bioabsorbable polymer.

The fixation member 115 can be formed as a retainer that is transferred through a tear 160 in tissue 165 and held at an outer surface 170 of the tissue 165 after deployment, as shown in FIGS. 1B and 1C.

The fixation member 115 can be formed as an anchor or a screw that is drilled or driven into the tissue during deployment, as shown in FIG. 15 of U.S. application Ser. No. 09/704,926. In an anchor or screw form, the fixation member 115 can include one or more threads on its outer surface to facilitate holding of the fixation member 115 to the tissue. Such anchor or screw forms are particularly adapted for use in hard tissue such as bone. The fixation member 115 can be formed with a generally cylindrical shape for receipt within a delivery device, such as a needle. The fixation member 115 can have a fin extending from its generally cylindrical shape.

The tissue repair device 100 also includes a flexible member 140, for example, a suture, that traverses the loop 105. As shown in FIGS. 1A and 1B, the flexible member 140 traverses the loop 105 by being passed through an interior 145 of the loop 105 that is bounded by or enclosed by the loop 105 and the fixation member 115. The flexible member 140 is a material suitable for implantation into hard or soft human tissue and it may be absorbable or nonabsorbable in the tissue after implantation. For example, the flexible member 140 can be made of a natural material, such as, for example, collagen, surgical silk, surgical cotton, or surgical steel. As another example, the flexible member 140 can be made of a synthetic material, such as, for example, a polymer or nylon.

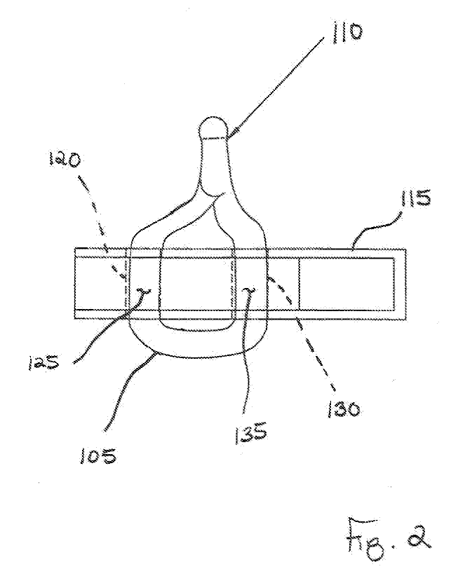

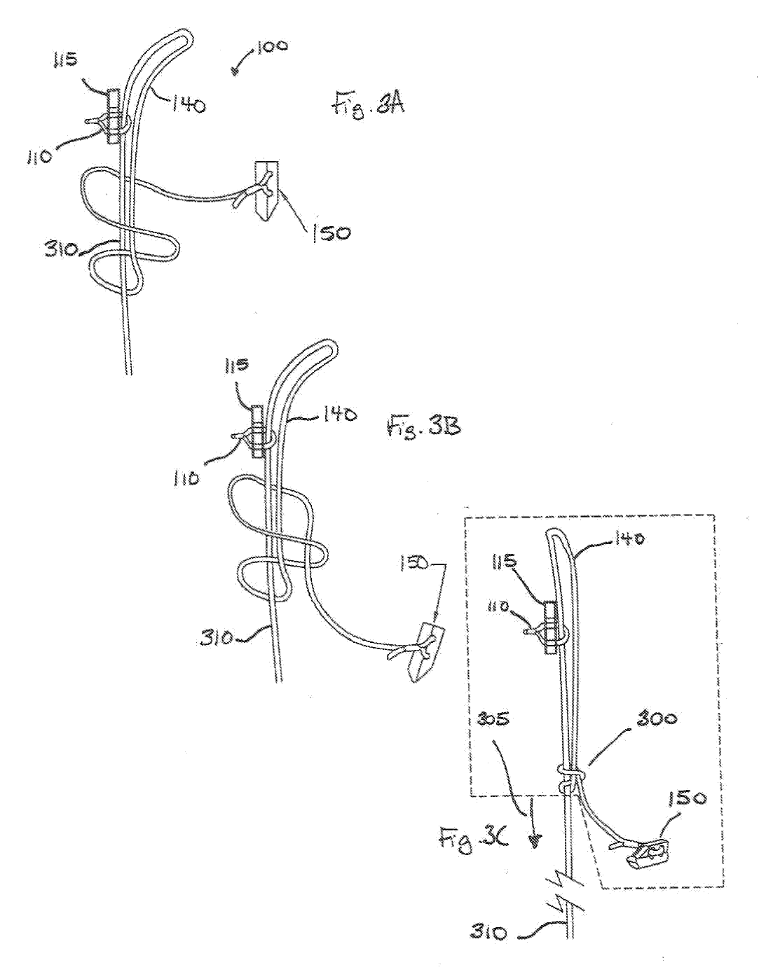

Referring also to FIGS. 3A-3C, the tissue repair device 100 can include a second fixation member 150 through which the flexible member 140 is passed, and a retaining element 300, for example, a slip knot in flexible member 140. The flexible member 140 is passed through the fixation member 150 by threading the flexible member 140 through a hole within the fixation member 150 and then attaching an end of the flexible member 140 to a region of the flexible member 140 that has not been threaded through the fixation member 150. The retaining element 300 permits the flexible member 140 to be pulled in the direction of arrow 305 and pass through the retaining element 300, thus reducing the distance between the fixation member 115 and the fixation member 150 and causing sides of the tear 160 to come into contact with each other. The retaining element 300 prevents an increase in distance between the fixation member 115 and the fixation member 150 to prevent the sides of the tear 160 from coming apart after coming in contact with each other.

Examples of the fixation members 115, 150, the retaining element 300, and the flexible member 140 can be found in U.S. application Ser. No. 10/918,445, filed Aug. 16, 2004, which is incorporated herein by reference.

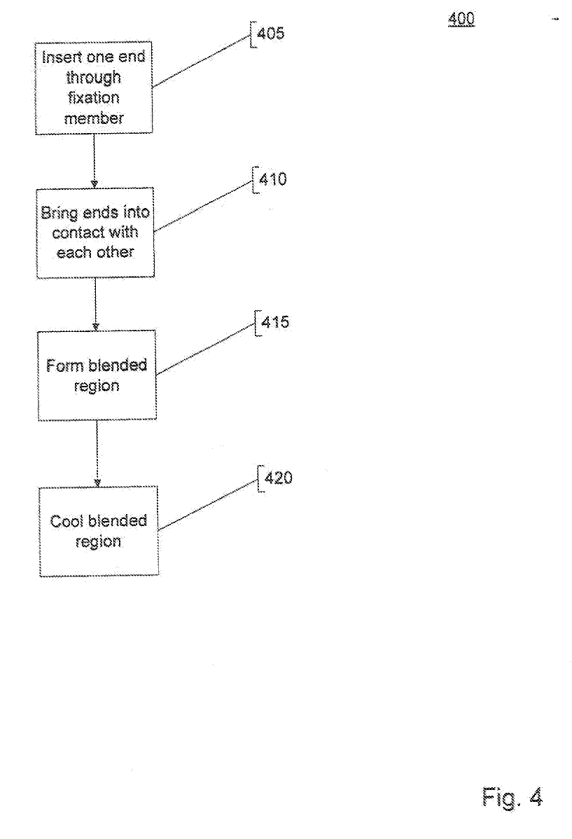

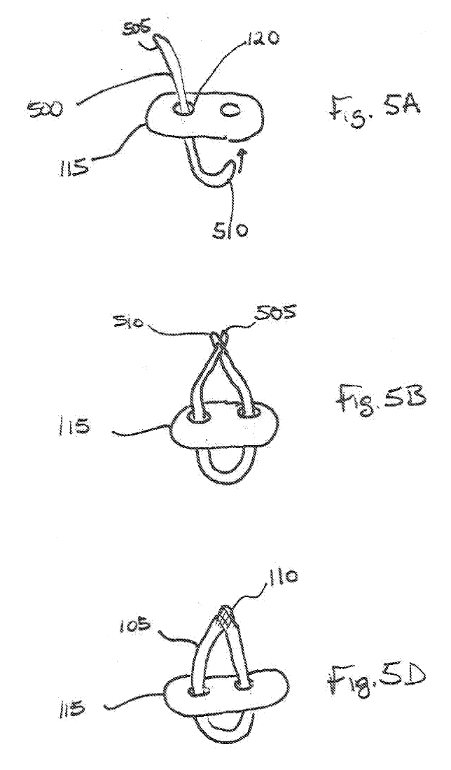

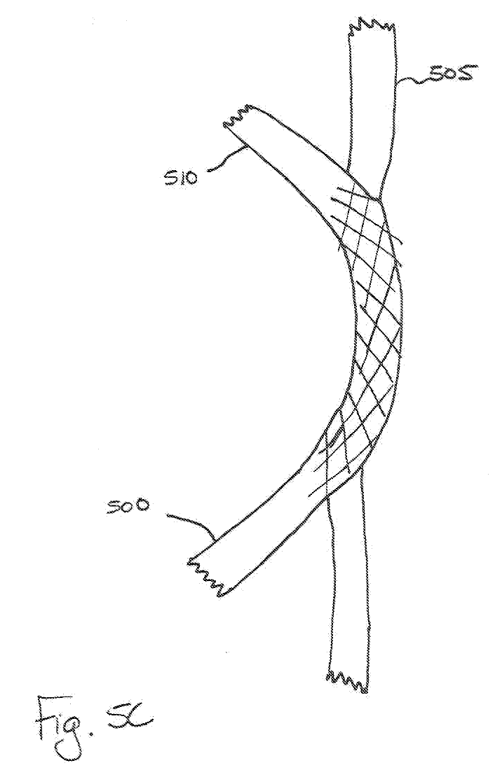

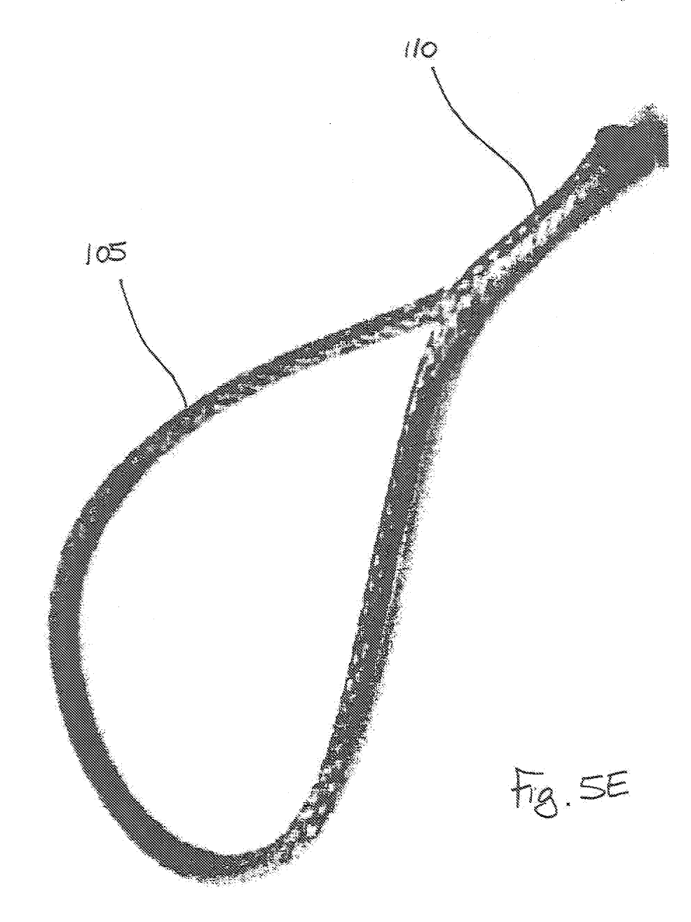

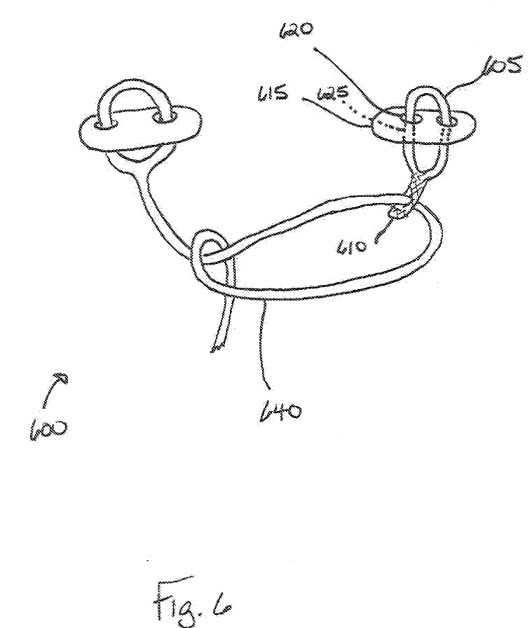

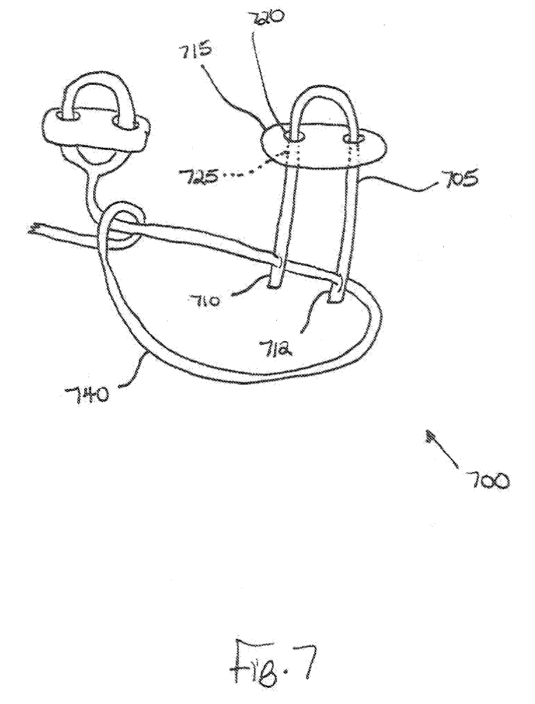

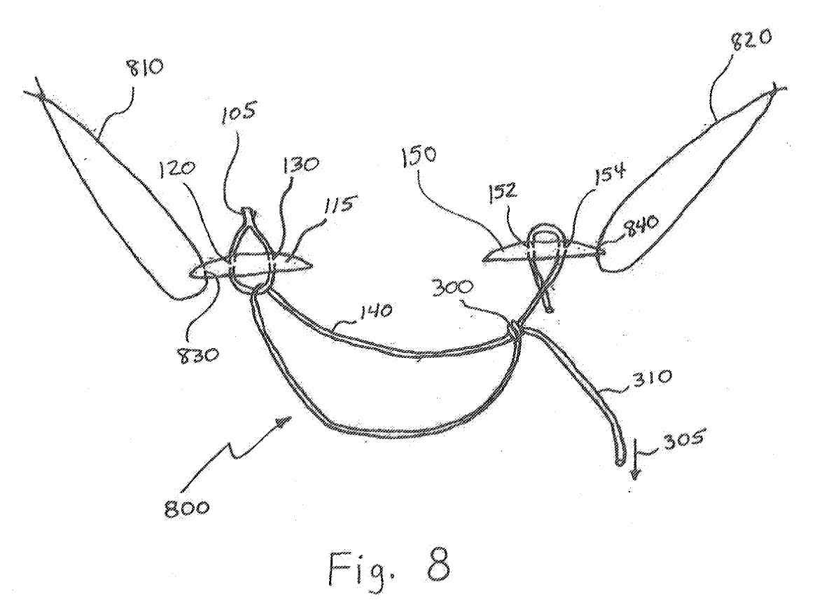

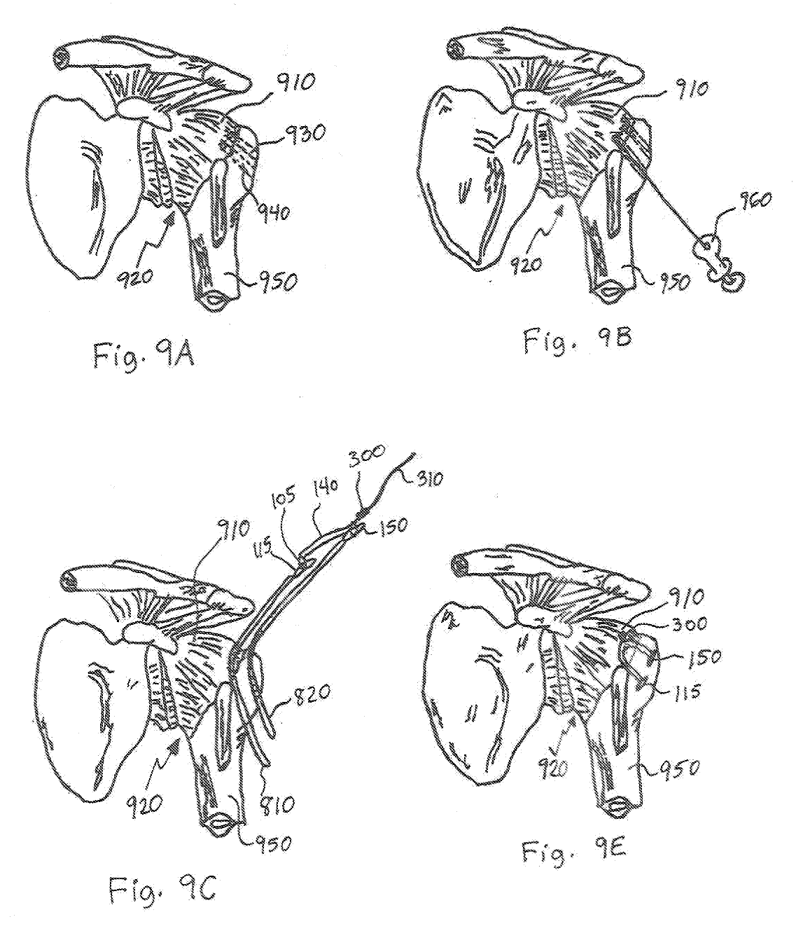

Referring to FIGS. 4 and 5A-5E, a procedure 400 is performed to form the loop 105. Initially, a first end 510 of the multifilament flexible material 500 is inserted or passed through the cavity 120 of the fixation member 115 (step 405). If desired, the multifilament flexible material 500 can be inserted through the second cavity 130 of the fixation member fixation member 115. After insertion, the first end 510 of the material 500 is brought into contact with a second end 505 (step 410). To facilitate thermal fusion, the ends 505, 510 can be interwoven into each other to make contact, as shown in FIG. 5B. In this case, the fibers of the end 505 are interwoven with the fibers of the end 510. For example, the end 505 can be inserted between fibers of the end 510, as shown in FIG. 5B. As another example, the end 505 can be inserted through an interior of a Chinese trap formed at the end 510, as shown in FIG. 5C.

Next, energy is supplied to the ends 505, 510 until the temperature of the ends 505, 510 raises to the point that the material in the ends 505, 510 melts or liquefies (step 415). At this point, the ends 505, 510 blend together to form a blended region, that is, a uniform or homogenous composition. Energy is supplied to the ends 505, 510 using, for example, thermal energy, ultrasonic energy, laser light, or electrical arc discharge. The ends 505, 510 can be inserted in a suitable energy supplying apparatus, depending on the way in which energy is provided to the ends 505, 510. For example, if the energy supplied is thermal energy, the ends 505, 510 can be locally heated using a heater element such as an electrical resistance heater element in the form of a thin film of an alloy. The heater element can create heat by other means, such as by induction, irradiation, or a chemical reaction. The blended region is allowed to cool to form a solid blended composition in the contact portion 110 (step 420).

The multifilament flexible material can be any material that is able to melt or liquefy upon application of an energy that raises its temperature and to solidify upon cooling such that the multifilament flexible material forms a blended region. Examples of materials having these properties include nylon, metals (such as titanium or steel), and polymer-based compounds, such as polyester fiber, polypropylene, polybutester, polyglactin, poliglecaprone, and polydioxanone. Another material that may have these properties is natural silk protein produced by spiders. The multifilament flexible material 500 can be any length and diameter that enables passage through the fixation member 615 and subsequent thermal fusion. For example, in one implementation in which the flexible material 500 is a type 0 size, the material 500 is about 4-12 mm long and has a diameter of about 0.4 mm.

The procedure 400 produces a contact portion 110 that has a yielding strength that is equivalent to or near to the United States Pharmacopoeia (USP) Standards value for a particular size of suture. For example, for a USP type 0 size suture, the yielding strength of the contact portion is about 12-13 pounds.

Referring to FIG. 6, in another implementation, a tissue repair device 600 includes a closed loop 605 of multifilament flexible material, similar in design to the loop 105 described above. The loop 605 is closed, thus, a first end of the multifilament flexible material contacts a second end of the multifilament flexible material to form a contact portion 610. One or more of the ends of the multifilament flexible material may include a Chinese trap.

The tissue repair device 600 also includes a fixation member 615 defining a cavity 620 that receives a part 625 of the loop 605, as discussed above with respect to FIG. 2. The tissue repair device 600 also includes a flexible member 640 that traverses the loop 605. As shown, the flexible member 640, in this implementation, traverses the loop 605 by passing through the contact portion 610 of the multifilament flexible material rather than passing through the interior of the loop 605. In this way, the flexible member 640 freely moves through the contact portion 610. For example, if the contact portion 610 includes a Chinese trap, then the flexible member 640 would pass directly through the Chinese trap.

Referring again to FIGS. 1B and 1C, the loop 105, 605 acts like a pulley through which the flexible member 140, 640 can freely slide to facilitate deployment of the fixation member 115, 615 into tissue 165. The pulley design reduces pinching of the flexible member 140, 640 between the surface 170 of the tissue 165 and the fixation member 115, 615 during deployment. Additionally, the loop 105 reduces friction between the flexible member 140, 640 and the fixation member 115, 615, thus enabling the flexible member 140, 640 to slide without being impeded by the edges of the fixation member 115, 615 or by the tissue 165 when the fixation member 115, 615 is deployed in tissue 165. Other pulley designs are shown in U.S. application Ser. No. 09/704,926. The device 100 or 600 can be delivered to the tissue 165 using a delivery device, such as, for example, the delivery devices shown in FIGS. 3, 5, 6, and 8-11 of U.S. application Ser. No. 09/704,926.

Referring to FIG. 7, in another implementation, a tissue repair device 700 includes a multifilament flexible element 705 having a thermally fused end 710 and a part 725 that is within a cavity 720 defined by a fixation member 715. Unlike the ends 505, 510 of the multifilament flexible material of the loop 105, the end 710 is thermally fused without being contacted to a second end 712 of the element 705. In this implementation, energy is supplied to the end 710 until the temperature of the end 710 raises to the point that the material in the end 710 melts or liquefies and blends together to form a blended, uniform composition. Energy may be supplied in any one of the manners mentioned above. Next, the blended composition at the end 710 is allowed to cool to form a solid blended composition.

The multifilament flexible element 705 can be any length and diameter that facilitates passage through the fixation member 715 and subsequent thermal fusion of the end 710. For example, in one implementation in which the flexible material 705 is a type 0 size, the material 500 is about 4-12 mm long and has a diameter of about 0.4 mm.

The tissue repair device 700 includes a flexible member 740 that is passed at least partially through the thermally fused end 710 by, for example, threading the flexible member 740 through the end 710 using a needle. After the flexible member 740 is passed through the end 710, it is free to move relative to the end 710. Thus, the multifilament flexible element 705 acts like a pulley through which the flexible member 740 can freely slide to facilitate deployment of the fixation member 715 into tissue.

To improve pullout strength between the flexible member 740 and the flexible element 705, the second end 712 of the element 705 can also be thermally fused (as discussed above with respect to the end 710) and the flexible member 740 can be passed through the thermally fused end 712, as shown.

Referring to FIG. 8, in another implementation, an apparatus for tissue repair 800 includes a first fixation member 115, a second fixation member 150, and a flexible coupling member 140 coupling fixation member 115 to fixation member 150. Apparatus 800 also includes flexible pull members 810, 820 attached to fixation member 115, 150, respectively. Flexible members 140, 810, and 820 are, for example, sutures. Fixation members 115, 150 include a plurality of openings, holes, or cavities 120, 130, and 830, and 152, 154, and 840, respectively, defined therein.

A loop 105, supra, passes through openings 120, 130 formed in fixation member 115. Although loop 105 is illustrated and discussed herein, other loop configurations, for example, loop 605 and element 705 may be used. As shown in FIG. 8, flexible coupling member 140 traverses loop 105 by being passed through an interior 145 of loop 105. Flexible coupling member 140 also traverses fixation member 150 through openings 152, 154 and forms a closed loop in the manner described above. Flexible coupling member 140 includes a slip knot 300. Slip knot 300 permits flexible coupling member 140 to be pulled in the direction of arrow 305 and pass through slip knot 300, thus reducing the distance between fixation member 115 and fixation member 150.

Flexible pull members 810, 820 are attached to fixation members 115, 150, respectively, by passing through openings 830, 840, respectively. As illustrated, flexible pull members 810, 820 are formed in a closed-loop by, for example, tying the ends of members 810, 820 together, though flexible pull members 810, 820 need not form closed loops.

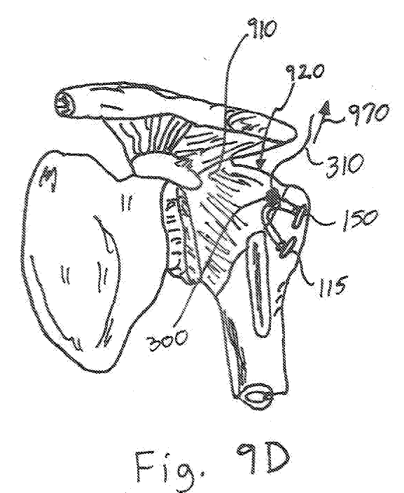

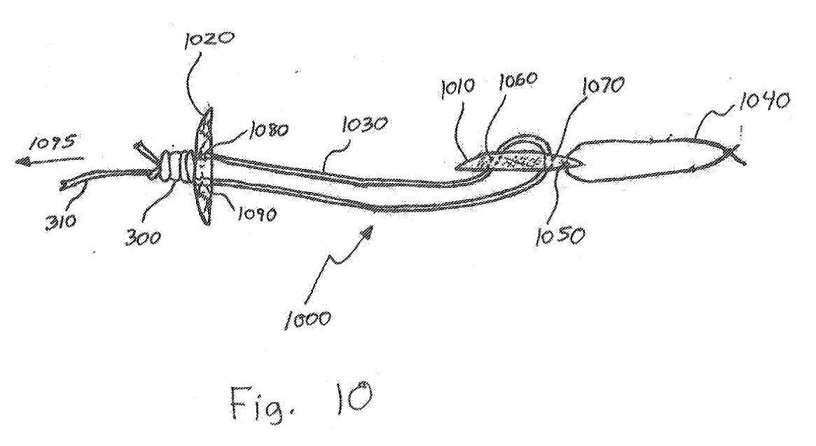

Referring to FIGS. 9A-9E, the apparatus 800 can be used, for example, under arthroscopic guidance, to repair a torn rotator cuff 910 in the shoulder joint 920. Referring to FIG. 9A, the physician initially forms at least two trans-osseous channels, 930, 940, through the humeral bone 950 using a drill (not shown) as described, for example, in U.S. patent application Ser. No. 10/918,445, supra. Referring to FIG. 9B, the physician pierces the rotator cuff tissue 910 by passing a needle 960, such as the needles shown in FIGS. 3, 5, 6, and 8-11 of U.S. application Ser. No. 10/918,445, through each of the trans-osseous channels 930, 940 and through the tissue 910. After piercing the tissue, the physician then grasps flexible pull members 810, 820, using, for example, needle 960, and advances each of the members 810, 820 and fixation members 115, 150 coupled, respectively, thereto, through the incisions made in the rotator cuff 910 and the trans-osseous channels 930, 940, as illustrated in FIG. 9C. Once fixation members 115 and 150 have exited channels 930, 940, and because of the way fixation members 115 and 150 are designed and configured, the fixation members 115, 150 rotate or toggle as they are pulled retrograde. This orientation helps to reduce the possibility of fixation members 115 and 150 unintentionally being pulled back into channels 930, 940.

Following placement of fixation members 810, 820 against the humeral bone tissue 950, flexible pull members 810, 820 are cut and removed. In addition, the physician pulls the trailing edge 310 of flexible member 140 in the direction of arrow 970 (FIG. 9D), reducing the distance between fixation member 115 and fixation member 150 and causing the soft tissue of the rotator cuff 910 to come into contact with the humeral bone tissue 950 thereby closing the wound. The slip knot 300 limits any tendency of the length of the flexible member 140 between fixation member 115 and fixation member 150 to increase. To complete the procedure, the physician cuts the trailing end 310 of flexible member 140 adjacent slip knot 300. The above-described method may also be used to close a wound in, for example, the soft tissue of the rotator cuff 910, as illustrated, for example, in FIGS. 1B and 1C.

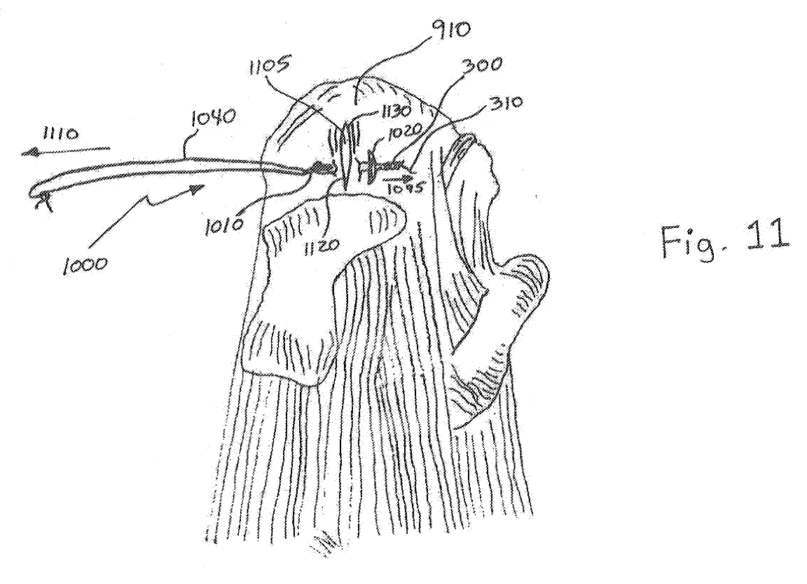

Referring to FIG. 10, in another implementation, an apparatus for tissue repair 1000 includes a first fixation member 1010, a second fixation member 1020, and a flexible coupling member 1030 coupling fixation member 1010 to fixation member 1020. Apparatus 1000 also includes a flexible pull member 1040 attached to fixation member 1010. Flexible members 1030 and 1040 are, for example, sutures. Fixation members 1010, 1020 include a plurality of openings, holes, or cavities 1050, 1060, and 1070, and 1080 and 1090, respectively, defined therein.

As shown in FIG. 10, flexible coupling member 1030 traverses fixation member 1010 through openings 1060, 1070 and traverses fixation member 1020 through openings 1080, 1090. Flexible coupling member 1030 includes a slip knot 300. Slip knot 300 permits flexible coupling member 1030 to be pulled in the direction of arrow 1095 and pass through slip knot 300, thus reducing the distance between fixation member 1010 and fixation member 1020.

Flexible pull member 1040 is attached to fixation member 1010 by passing through opening 1050. As illustrated, flexible pull member 1040 is formed in a closed-loop by, for example, tying the ends of member 1040, though flexible pull member 1040 need not form a closed-loop.

Referring to FIG. 11, the apparatus 1000 can be used, for example, under arthroscopic guidance, to repair a tear 1105 in the soft tissue of a rotator cuff 910. The physician initially forms a channel (not shown) by passing a needle, such as the needles shown in FIGS. 3, 5, 6, and 8-11 of U.S. application Ser. NO. 10/918,445, through a portion of the rotator cuff tissue 910 on either side of, and through, tear 1105. As illustrated in FIG. 11, the physician then advances fixation member 1010 through the channel formed in tissue 910 and across tear 1105 by pulling flexible pull member 1040 in the direction of arrow 1110. This also pulls flexible member 1030 across tear 1105 and through the channel. After fixation member 1010 has exited the channel and is positioned on a side 1120 of tear 1105, because of the manner in which fixation member 1010 is designed and configured, fixation member 1010 rotates or toggles as it is pulled retrograde. This orientation helps to reduce the possibility of fixation member 1010 unintentionally being pulled back into the channel. As illustrated in FIG. 11, when fixation member 1010 is positioned proximate side 1120 of tear 1105, flexible member 1030 traverses tear 1105 and fixation member 1020 is positioned proximate side 1130 of tear 1105.

Once fixation members 1010, 1020 are in position on either side (1120, 1130, respectively) of tear 1105, the physician cuts and removes flexible pull member 1040. In addition, the physician pulls the trailing edge 310 of flexible member 1030 in the direction of arrow 1095 to allow the flexible member to pass through slip knot 300, thus reducing the distance between fixation members 1010 and 1020 and causing the sides (1120, 1130) of tear 1105 to come into contact with each other, thereby closing the wound. The slip knot 300 limits any tendency of the length of the flexible member 1030 between fixation member 1010 and fixation member 1020 to increase. To complete the procedure, the physician cuts the trailing end 310 of flexible member 1030 adjacent slip knot 300.

A number of embodiments of the invention have been described. Nevertheless, it will be understood that various modifications may be made without departing from the spirit and scope of the invention. For example, the multifilament flexible material or the contact portion may include a growth factor, such as, for example, an angiogenic factor. The multifilament flexible material or the contact portion may be loaded with a bioactive material, a stimulant, or any substance that promotes healing of the tissue.

As another example, the contact portion can be formed by stitching the ends of the multifilament flexible material together without raising the temperature at the ends by using an additional element of similar ligature as the thread. For example, if the multifilament flexible material is a type 0 size, then the thread can be a high strength polyethylene suture of 2-0, 4-0, or 8-0 size using the USP standards.

As another example, in the implementation illustrated in FIG. 8, flexible member 140 could traverse fixation member 115 directly through, for example, openings 120, 130, in the case where, for example, loop 105 is not present in the apparatus 800. Moreover, in the implementation illustrated in FIGS. 9A-9E, each of the steps of creating a channel, piercing the tissue, and advancing the fixation members 115, 150 through the channels may be performed simultaneously or one-at-a-time for each respective fixation member 115, 150. Accordingly, other embodiments are within the scope of the following claims.

What is claimed is:

1. An apparatus for tissue repair, comprising:
first and second fixation members configured to secure tissue together;
a flexible coupling member coupling the first and second fixation members;
a first flexible pull member attached to the first fixation member but not the second fixation member; and
a second flexible pull member attached to the second fixation member but not the first fixation member.

2. The apparatus of claim 1, wherein the flexible coupling member includes a slip knot.

3. The apparatus of claim 2, wherein the flexible coupling member includes a first closed loop.

4. The apparatus of claim 3, wherein the first and second fixation members each define at least one opening.

5. The apparatus of claim 4, wherein the first closed loop traverses the opening in one of the fixation members.

6. The apparatus of claim 5, further comprising:
a second closed loop of flexible material traversing the opening in the other fixation member and the flexible coupling member being slidably received through the second closed loop.

7. The apparatus of claim 1, wherein the first and second flexible pull members comprise closed loops.

8. The apparatus of claim 1, wherein the flexible coupling member and the first and second flexible pull members comprise sutures.

9. A surgical method for repairing a wound in a rotator cuff comprising:
forming a first channel through tissue;
advancing a first flexible member and a first fixation member coupled thereto through the first channel;
forming a second channel through tissue;
advancing a second flexible member and a second fixation member coupled thereto through the second channel; and
pulling a third flexible member coupling the first and second fixation members to shorten a length of the third flexible member between the first and second fixation members.

10. The method of claim 9, wherein the first and second channels are formed through rotator cuff soft tissue.

11. The method of claim 9, wherein the first and second channels are formed through bone tissue.

12. A surgical method, comprising:
pulling a first implant through soft tissue of a rotator cuff across a tear in the tissue to position the first implant on a first side of the tear and a second implant, coupled to the first implant by a flexible coupling member, on a second side of the tear with the flexible coupling member traversing the tear; and
pulling the flexible coupling member to shorten a length of the flexible coupling member between the first and second implants to move the implants against the soft tissue to close the tear.

13. The method of claim 12, further comprising:
forming a channel through the soft tissue and across the tear.

14. The method of claim 13, wherein pulling the first implant includes pulling a first closed loop of flexible material attached to the first implant through the channel.

15. A method of making a tissue repair device, the method comprising:
passing a flexible material through an opening defined by a first fixation member;
forming the flexible material into a first closed loop;
traversing a flexible member through the first closed loop;
traversing the flexible member through a second fixation member and forming a second closed loop in the flexible member such that the flexible member is coupled to the second fixation member; and
forming a slip knot in the flexible member.

16. The method of claim 15, further comprising:
coupling a first flexible pull member to the first fixation member but not the second fixation member; and
coupling a second flexible pull member to the second fixation member but not the first fixation member.

17. A tissue repair device, comprising:
a closed loop of multifilament flexible material, wherein the loop is knotless and includes a contact portion in which the ends of the multifilament flexible material are formed together; and
a fixation member defining a cavity that receives at least a part of the closed loop.
